(19) United States
(12) Patent Application Publication
(10) Pub. No.: US 2023/0414959 A1
(43) Pub. Date: Dec. 28, 2023

(54) METHODS AND SYSTEMS FOR TREATMENT OF NEUROPSYCHIATRIC DISORDERS

(71) Applicant: Magnus Medical, Inc., Burlingame, CA (US)

(72) Inventors: Shan H. SIDDIQI, Hoffman Estates, IL (US); Adi MARON-KATZ, Binyamina-Giv'at Ada (IL); Armani PORTER, Pikesville, MD (US); Brandon S. BENTZLEY, Half Moon Bay, CA (US); Brett M. WINGEIER, San Francisco, CA (US)

(21) Appl. No.: 18/117,351

(22) Filed: Mar. 3, 2023

Related U.S. Application Data
(60) Provisional application No. 63/316,330, filed on Mar. 3, 2022.

(51) Int. Cl. A61N 2/00; A61N 2/02; A61M 21/02; G16H 20/70

(52) U.S. Cl. CPC A61N 2/006; A61N 2/02; A61M 21/02; A61N 2/002; G16H 20/70; A61M 2021/0055

(57) ABSTRACT

Described herein are methods and systems for treatment of neurological conditions using aTBS. One variation includes a method for treating anxiety disorders, including generating a personalized accelerated theta burst stimulation (aTBS) target, placing a TMS device relative to the patient's brain such that the focus of the magnetic field is over the aTBS target, applying TMS to the aTBS target according to an aTBS protocol to the brain using the TMS device to alleviate one or more symptoms of the anxiety disorder. Methods and systems for concurrently treating two or more disorders, e.g., depression and an anxiety disorder, are also described.

### CROSS-REFERENCE TO RELATED APPLICATIONS

This application claims priority to U.S. Provisional Application No. 63/316,330, filed on Mar. 3, 2022, which is hereby incorporated by reference in its entirety.

### TECHNICAL FIELD

This application generally relates to methods for treating neuropsychiatric disorders, including anxiety disorders such as generalized anxiety disorder and anxiety-related disorders such as post-traumatic stress disorder (PTSD) and obsessive-compulsive disorder (OCD). The methods may include determining personalized targets for neuromodulation based on functional connectivity data, clinical symptoms, and/or other clinical factors of the subject such as level or duration anxiety, and if more than one disorder is present, concurrent treatment of the disorders. Systems for determining the personalized targets and concurrently treating neuropsychiatric disorders are also described herein.

### BACKGROUND

According to the U.S. National Institutes of Mental Health (NIMH), generalized anxiety disorder (GAD) is the most common psychiatric disorder in the United States. Individuals experiencing GAD have excessive anxiety and worry about several areas of life for at least six months. For some individuals, GAD may result in severe impairments that interfere with or limit their ability to carry out major life activities. Although treatments exist for GAD, the condition tends to be chronic and highly treatment resistant. For example, in a 12-year longitudinal study, about 73% of people treated for GAD were found to relapse (Bruce, S. E., Yonkers, K. A., Otto, M. W., Eisen, J. L., Weisberg, R. B., Pagano, M., Shea, M. T., & Keller, M. B. (2005). Influence of Psychiatric Comorbidity on Recovery and Recurrence in Generalized Anxiety Disorder, Social Phobia, and Panic Disorder: A 12-Year Prospective Study. American Journal of Psychiatry, 162(6), 1179-1187).

Transcranial Magnetic Stimulation (TMS) is a non-invasive medical procedure where strong magnetic fields are utilized to stimulate specific areas of an individual's brain to treat medical conditions such as depression and obsessive-compulsive disorder (OCD). When TMS is repeatedly applied in a short time frame, it is referred to as repetitive TMS (rTMS). Accelerated theta-burst stimulation (aTBS) is a patterned form of rTMS, typically administered as a triplet of stimulus pulses with 20 ms between each stimulus in the triplet (therefore having a pulse frequency of 50 Hz), where the triplet is repeated every 200 ms (therefore having triplets, or bursts, occurring at a frequency of 5 Hz), although other combinations of pulse and burst timing may also be used. Although a few studies have used rTMS to treat GAD, none were designed to examine the dose, pattern of stimulation, and/or the specific stimulation target linked to clinical improvement. Accordingly, methods and systems that assess one or more of these factors would be beneficial.

### SUMMARY

Described herein are methods and systems for treating neuropsychiatric disorders, including GAD and/or other anxiety disorders, that may help achieve desired clinical results by personalizing the brain target for neurostimulation and/or the pattern of stimulation which is delivered. Such neuropsychiatric disorders may be network disorders, in which symptoms are related to relative excitation and/or inhibition of one or more interconnected regions of the brain. The methods and systems may treat a single neuropsychiatric disorder, or may treat two or more neuropsychiatric disorders concurrently.

Personalization of the brain target may include obtaining data related to one or more symptoms experienced in addition to anxiety and/or one or more clinical factors from the subject when determining the brain target. Personalization may also include obtaining data related to brain connectivity and brain activity in order to locate a brain target that is optimally connected to one or more other regions, and/or which is optimally positioned for stimulation. For any given disorder, the neurostimulation target may reside within either the left hemisphere or the right hemisphere of the brain, but for anxiety and related disorders such as GAD or PTSD, it may be beneficial for the right hemisphere to be stimulated. In some instances, for example, when the patient has been diagnosed with both depression and anxiety or an anxiety-related disorder, it may be useful to target neurostimulation therapy to one or more regions within the left hemisphere for depression, and to one or more regions within the right hemisphere for anxiety or an anxiety-related disorder. The one or more targeted regions in the left hemisphere may be related to one or more symptoms of depression. Similarly, the one or more targeted regions in the right hemisphere may be related to one or more symptoms of anxiety.

In general, the methods for treating an anxiety disorder may include selecting a region of interest within the brain of the subject, measuring or identifying one or more symptoms experienced by the subject in addition to anxiety, determining a neurostimulation target in the right hemisphere of the brain based on the one or more symptoms and functional neuroimaging data, and applying magnetic stimulation to the neurostimulation target. The one or more symptoms experienced in addition to anxiety may be, for example: anhedonia, depression, dysphoria, panic, fear, fatigue, insomnia, obsessive-compulsiveness, restlessness, weight change, cognitive change, mood, tension, somatic sensations such as dizziness or numbness, somatic muscular events such as twitching, cardiovascular symptoms such as tachycardia, respiratory symptoms such as constriction in the chest, gastrointestinal symptoms such as looseness of bowels, genitourinary symptoms such as impotence or loss of libido, autonomic symptoms such as dry mouth, feelings of irritation, feelings of guilt, or other symptoms of depression and/or anxiety, or combinations thereof.

Additionally, or instead of measuring or identifying one or more symptoms, the method may include measuring or identifying one or more clinical factors about the subject to determine the neurostimulation target. Such clinical factors may include, for example: duration of anxiety, severity of anxiety, treatment history for an anxiety disorder, family history of an anxiety disorder, family history of a depressive disorder, post-traumatic stress disorder, an eating disorder, fibromyalgia, demographic information, history of OCD, history of panic attacks, history of major depression, history of substance abuse, history of comorbid pain, other correlates or comorbidities of depression and/or anxiety, or combinations thereof.

Additionally, or instead of measuring or identifying symptoms and/or clinical factors, the method may include measuring biosignals related to symptoms of an anxiety or other neuropsychiatric disorder, and/or related to the effects of neuromodulation, such as electroencephalographic (EEG) data, functional near-infrared spectroscopy (fNIRS) data, electromyographic (EMG) data, electrical impedance plethysmography, galvanic skin response (GSR), or actigraphy. For example, actigraphy may be used as a proxy for somatic symptoms in a treatment method. In some methods, the region of interest may include a deep brain structure, which because of its depth or shape, may be difficult to stimulate by non-invasive means. Thus, a neurostimulation target may be chosen that is functionally correlated with or functionally connected to the deep brain structure, in order for stimulation of the neurostimulation target to result in modulation of activity in the deep brain structure. For example, when treating generalized anxiety, the deep brain structure may be the amygdala, and the neurostimulation target may be the dorsolateral prefrontal cortex.

The systems for treating a neuropsychiatric disorder such as an anxiety disorder may include a transcranial magnetic stimulation coil, a data module configured to receive data comprising functional neuroimaging data of a brain of a subject, where the functional neuroimaging data describes neuronal activation within the brain, and a targeting module comprising a memory storing a set of instructions. The targeting module may include one or more processors responsive to the set of instructions that are configured to determine a neurostimulation target in a hemisphere of the brain such as the right hemisphere based on one or symptoms experienced by the subject, and/or functional neuroimaging data. The one or more symptoms are generally those experienced in addition to anxiety. The transcranial magnetic stimulation coil may be configured to apply aTBS to the neurostimulation target. In some variations, the aTBS may be accelerated intermittent Theta-Burst Stimulation (aiTBS). In other variations, the aTBS may be accelerated continuous Theta-Burst Stimulation (acTBS).

### BRIEF DESCRIPTION OF THE DRAWINGS

FIG. 1 depicts a diagram of an exemplary accelerated Theta-Burst Stimulation (aTBS) system for delivering neurostimulation to a personalized target.

FIG. 2 depicts a diagram of an exemplary accelerated Theta-Burst Stimulation (aTBS) computing system.

FIG. 3 depicts another exemplary system for delivering neurostimulation to a personalized target.

FIG. 4 is a flowchart depicting an exemplary method for determining personalized targets for treating anxiety disorders including steps of obtaining symptoms from the subject and weighting different targets based on the symptoms.

FIG. 5 is a flowchart depicting another exemplary method for determining personalized targets for treating anxiety disorders including steps of obtaining symptoms from the subject and determining a target based on the symptoms.

FIG. 6 is a flowchart depicting yet another exemplary method for determining personalized targets for treating anxiety disorders based on connectivity values for various brain locations.

FIG. 7 depicts yet another exemplary method for determining personalized targets for treating anxiety disorders based on identifying a peak connectivity of a sub-parcel from a plurality of sub-parcels.

FIG. 8 illustrates the process of data collection in a patient study for determining personalized targets for treating anxiety disorders.

FIG. 9 is a flowchart depicting an exemplary method for determining personalized targets to concurrently treat two disorders.

FIG. 10 is a flowchart depicting an exemplary method for determining personalized targets for concurrent application of stimulation to treat a neuropsychiatric condition.

### DETAILED DESCRIPTION

Described herein are methods and systems for determining personalized targets in the brain for neuromodulation treatment of neuropsychiatric disorders such as anxiety and related disorders. By personalizing neurostimulation targets, the methods and systems may stimulate surface regions of the brain that are linked to deep regions, thereby bypassing the need for deep brain stimulation. Furthermore, personalization of neurostimulation targets may reduce the treatment time required to achieve desired clinical results and/or impart longer lasting beneficial effects on the treated subject. In addition to assessing the functional connectivity of various neurostimulation targets to deep brain structures, symptoms that the subject may be experiencing in addition to anxiety may be used to help determine the neurostimulation targets. Clinical factors such as the level or duration of anxiety associated with the subject may also be included in the assessment to help determine the neurostimulation targets.

The described methods and systems may be used to treat various anxiety disorders and anxiety-related disorders, including without limitation, generalized anxiety disorder, panic disorder, PTSD, OCD, and phobias such as social phobia or agoraphobia. The described methods and systems may also be used to treat neuropsychiatric disorders including without limitation depression, pain disorders, or substance use disorders, or used to treat patients with coexisting or comorbid disorders including without limitation, depression coexisting with PTSD, or depression coexisting with anxiety. In some variations, the methods and systems may be used to treat two or more neuropsychiatric disorders concurrently. For example, the described methods and systems may be used with a stimulation target in the left hemisphere for the treatment of depression symptoms, and with a stimulation target in the right hemisphere for the treatment of anxiety symptoms.

### Methods

The methods for treating anxiety disorders described herein generally include selecting a region of interest within the brain of the subject, obtaining one or more symptoms from the subject, determining a neurostimulation target in the right hemisphere of the brain based on the one or more symptoms and functional neuroimaging data, and applying magnetic stimulation to the neurostimulation target. The one or more symptoms may be those experienced in addition to anxiety, and may include anhedonia, depression, dysphoria, panic, fear, fatigue, insomnia, obsessive-compulsiveness, restlessness, weight change, cognitive change, mood, tension, somatic sensations such as dizziness or numbness, somatic muscular events such as twitching, cardiovascular symptoms such as tachycardia, respiratory symptoms such as constriction in the chest, gastrointestinal symptoms such as looseness of bowels, genitourinary symptoms such as impotence or loss of libido, autonomic symptoms such as dry mouth, feelings of irritation, feelings of guilt, or other symptoms of depression and/or anxiety, or combinations thereof.

The region of interest in the brain may be associated with the regulation of mood. For example, the region of interest may be the amygdala, the hippocampus, or the dorsomedial thalamus. When anxiety is to be treated, the region of interest may be a deep brain structure such as the amygdala. One or more cortical regions of the brain may be functionally connected to the region of interest. For example, the region of interest may be functionally connected to any part of the frontal, temporal, parietal, and/or occipital cortex. In some variations, parts of the prefrontal cortex are functionally connected to the deep brain structure. For example, parts of the dorsolateral prefrontal cortex may be functionally connected to the amygdala, and used as a neurostimulation target to treat generalized anxiety. Other cortical regions of the brain may include the anterior dorsolateral prefrontal cortex, the dorsal anterior cingulate cortex, the inferior temporal cortex, the lateral parietal cortex, the middle cingulate cortex, the posterior cingulate cortex, the posterior dorsolateral prefrontal cortex, the posterior occipital cortex, the subgenual anterior cingulate cortex, or the ventrolateral prefrontal cortex. In some variations, the region of the brain that is functionally connected to a deep brain structure is the intraparietal sulcus, the pre/post central gyms, the superior parietal lobule, the superior temporal gyms, or the temporal-parietal junction. In some variations, the region of the brain that is functionally connected to a deep brain structure includes sub-regions that are connected in an excitatory manner or sub-regions that are connected in an inhibitory manner. In other variations, the sub-regions may not be functionally connected to the deep brain structure, but may be interspersed or adjacent with each other, and thus, the sub-region or sub-regions that can be stimulated using a non-invasive modality and yield a desired excitatory or inhibitory effect may be chosen as the personalized brain target. Functional connectivity may be identified using functional neuroimaging modalities such as functional magnetic resonance imaging (fMRI), or may be estimated using modalities such as diffusion tensor imaging (DTI).

The regions of interest in the brain may also comprise a brain network associated with the particular type of anxiety disorder. For example, the brain network may comprise the cingulo-opercular network, the fronto-parietal network, the dorsal attention network, the ventral attention network, the visual network, the sensorimotor network, the default mode network, the salience network, or the anxiosomatic circuit. In one variation, the brain network associated with the anxiety disorder is the dorsal attention network (DAN). In another variation, the brain network associated with the anxiety disorder is the anxiosomatic circuit.

Some variations of the method may further include measuring one or more clinical factors about the subject, and identifying the neurostimulation target based on the one or more symptoms, functional neuroimaging data, and one or more clinical factors. The one or more clinical factors may be duration of anxiety, severity of anxiety, treatment history for an anxiety disorder, family history of an anxiety disorder, family history of a depressive disorder, post-traumatic stress disorder, an eating disorder, fibromyalgia, demographic information, history of OCD, history of panic attacks, history of major depression, history of substance abuse, history of comorbid pain, other correlates or comorbidities of depression and/or anxiety, clinical data (e.g., heart rate, heart rate variability, temperature, or blood pressure), or combinations thereof. In one instance, the clinical factor is the severity of anxiety.

### Determination of Neurostimulation Targets

Every person has a unique brain structure and connectivity. The methods described herein generally use this unique brain structure and connectivity to personalize brain targets for neurostimulation treatment of anxiety disorders. The methods may employ one or more algorithms that combine functional neuroimaging data with one or more symptoms experienced by the subject in addition to anxiety. The functional neuroimaging data may include fMRI data. As previously mentioned, the one or more symptoms may be depression, panic, fear, fatigue, insomnia, obsessive-compulsiveness, or combinations thereof. The one or more symptoms may be obtained from a questionnaire filled out by the subjects, from a task battery completed by the subjects, and/or from an interview conducted by a psychiatrist or other trained evaluator. Depending on the symptoms being experienced by the subject, the algorithm may instruct for more than a single target to be stimulated. For example, when different symptoms are being experienced, or if multiple regions are identified having desired functional connectivity with a deep brain structure, the algorithm may instruct for different targets to be stimulated. In one variation, when depression is experienced with anxiety, the neurostimulation target may be the anxiosomatic circuit in the right superior frontal gyms. In general, for anxiety-related disorders, the neurostimulation targets will be located in the right hemisphere of the brain of the subject. One exemplary strategy for neurostimulation targets the cingulo-opercular network, and applies magnetic stimulation to the subregion of the right dorsolateral prefrontal cortex (R-DLPFC) having a maximum resting state fMRI anti-correlation with the amygdala. Another exemplary strategy for neurostimulation targets the anxiosomatic circuit, and applies magnetic stimulation to the R-DLPFC having a maximum resting state fMRI correlation with the anxiosomatic circuit.

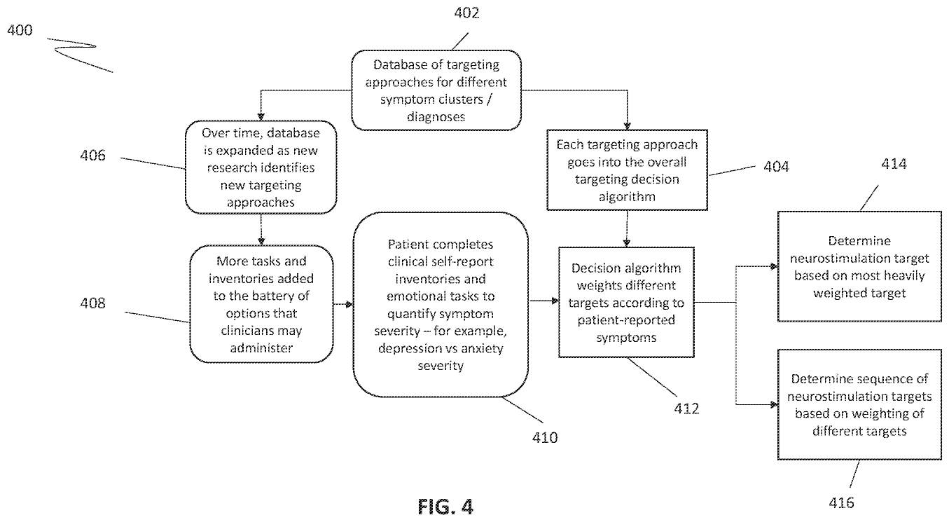

In general, the methods for determining neurostimulation targets described herein may s having the steps illustrated in FIGS. 4-7. In one example, as shown in FIG. 4, the algorithm (400) first includes the step of compiling a database of targeting approaches for different clusters (groups) of symptoms, clinical factors, and/or diagnoses (402). Next, each targeting approach may be input into the overall targeting decision algorithm (404). Over time, the database may be expanded as studies identify new targeting approaches (406), and more measurable items (based on tasks, such as cognitive or psychophysiological tasks scored on accuracy and/or reaction time, and/or questionnaires) are added to the battery of options that clinicians may administer (408). One clinician-administered inventory, or questionnaire, that may be used is the Montgomery-Asberg Depression Rating Scale (MADRS), a ten-item diagnostic questionnaire which clinicians use to measure the severity of depressive episodes in patients. Similarly, another inventory that may be used by clinicians may be the Hamilton Rating Scale for Anxiety (HAM-A). Based on the self-report completed by the subject that includes tasks and questionnaires that help quantify symptom severity (e.g., depression vs. anxiety severity) (410), the algorithm weights different neurostimulation targets according to the symptoms reported by the subject (412), and may determine a single neurostimulation target based on the most heavily weighted target (414) or may determine a sequence of neurostimulation targets based on the weighting of different targets (416), for example by recommending stimulation of the two or more most heavily-weighted targets, with stimulation intensity, duration, or number of sessions delivered to an individual target proportional to the weight of the individual target. One patient self-reported inventory may be the Generalized Anxiety Disorder-7 (GAD-7), a seven-item patient scale focused on generalized anxiety disorder (GAD) that uses some of the Diagnosis and Statistical Manual of Mental Disorders, Edition (DSM-5) criteria for GAD to identify probable cases of GAD along with measuring anxiety symptom severity.

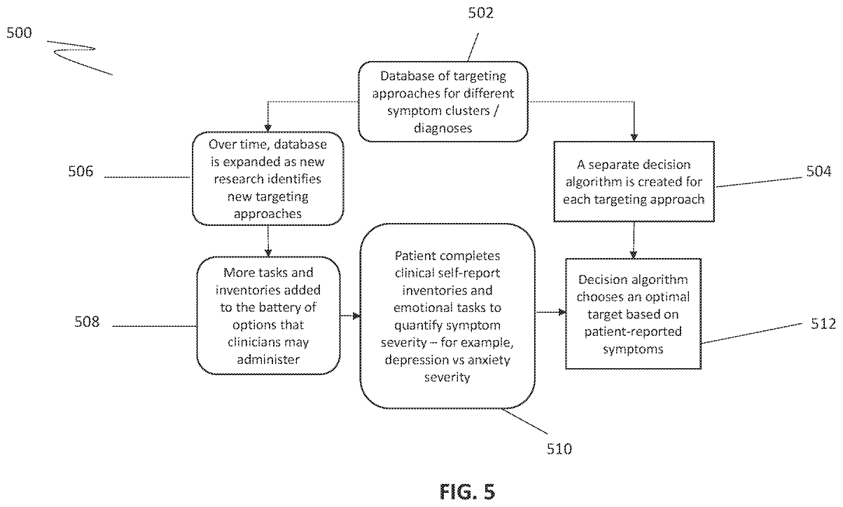

In another variation, as shown in FIG. 5, the algorithm (500) also includes the step of compiling a database of targeting approaches for different clusters (groups) of symptoms, clinical factors, and/or diagnoses (502). However, a separate decision algorithm may be created for each targeting approach (504). Over time, the database may be expanded as studies identify new targeting approaches (506), and more tasks, such as cognitive or psychophysiological tasks scored on accuracy and/or reaction time, and questionnaires are added to the battery of options that clinicians may administer (508). Based on the self-report and/or clinician assessment that includes tasks and questionnaires that help quantify symptom severity (e.g., depression vs. anxiety severity) (510), the algorithm may determine a neurostimulation target based on the symptoms reported by the subject (patient).

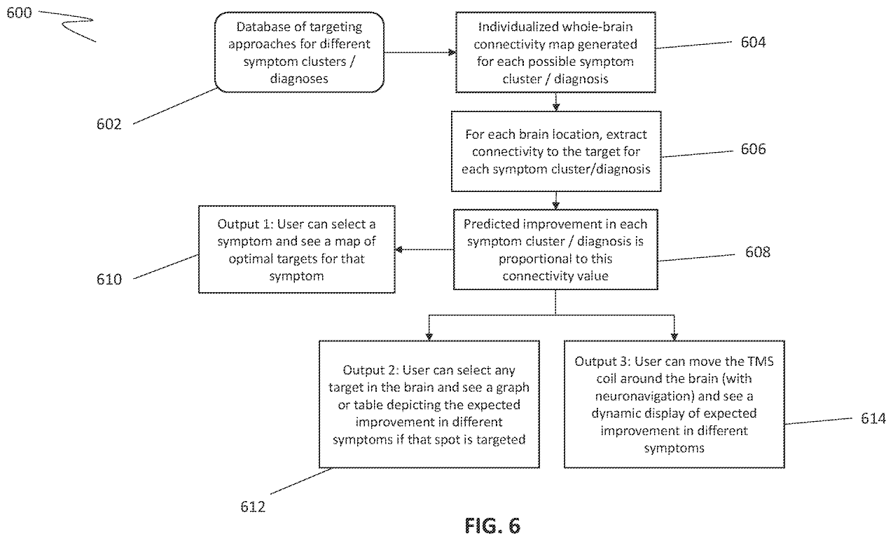

Referring to FIG. 6, yet another neurostimulation targeting algorithm is illustrated. In FIG. 6, the algorithm (600) first includes the step of compiling a database of targeting approaches for different clusters (groups) of symptoms, clinical factors, and/or diagnoses (602). An individualized whole-brain connectivity map may then be generated for each possible cluster of symptoms and/or diagnoses (604). Next, for each brain location, connectivity to the target may be extracted for each possible cluster of symptoms and/or diagnoses (606), and improvement in each cluster of symptoms and/or diagnoses predicted as being proportional to the connectivity value (608). Based on this connectivity value, the clinician may select three options. In the first option (610), the clinician may select a symptom and see a map of identified targets for that symptom. In the second option (612), the clinician may select a target in the brain and see a bar graph (or other visualization, such as a table) depicting the expected improvement in different symptoms if that area is targeted. With the third option (614), an aTBS device (e.g., a TMS coil) may be moved around the brain using neuronavigation, and expected improvements in different symptoms shown in real-time on a display that correlate with the brain target over which the TMS coil is positioned at any given moment.

Some variations of the method may include determining a neurostimulation target by dividing the region of interest (ROI) in the brain into a plurality of sub-parcels, and determining a peak connectivity site for each of the plurality of parcels. In these variations, information relating to symptoms or clinical factors may or may not be used in combination with peak connectivity to determine the neurostimulation target. The ROI may be divided into various amounts of sub-parcels, for example, between 2 and 100 sub-parcels, including all values and sub-ranges therein. For example, the ROI may be divided into 2, 5, 15, 20, 25, 30, 35, 40, 45, 50, 55, 60, 65, 70, 75, 80, 85, 90, 95, or 100 sub-parcels.

In one variation, the peak connectivity site may be characterized by a pattern of neuronal activity having a high degree of correlation to a pattern of neuronal activity in the corresponding sub-parcel. When a high degree of correlation is present, the ROI may include or be associated with the anxiosomatic circuit. In another variation, the peak connectivity site may be characterized by a pattern of neuronal activity having a high degree of anti-correlation to a pattern of neuronal activity in the corresponding sub-parcel. When a high degree of anti-correlation is present, the ROI may include or be associated with the amygdala. In yet further variations, determining the neurostimulation target may comprise selecting a subset of the plurality of peak connectivity sites that cluster within a common neuroanatomical region, and designating at least a portion of the common neuroanatomical region as the neurostimulation target.

Other variations of the method may include determining a neurostimulation target by assigning, for each of a plurality of potential target locations, a degree of connectivity to each of the plurality of sub-parcels; calculating, for each of the plurality of potential target locations, an overall degree of connectivity based on a numerical average of the degree of connectivity to each of the plurality of sub-parcels; and selecting one or more potential target locations having an overall degree of connectivity above a predetermined threshold as the neurostimulation target.

More specifically, when peak connectivities of sub-parcels are employed, the method may include a plurality of small neuroanatomical volumes (which may be referred to as “seeds”) that may be sub-parcels of a larger region of interest, rather than a single seed to determine a neurostimulation target. The use of a plurality of ROI sub-parcels as seeds may obviate the need to rely on assuming that a specific seed may be localized in an individual subject, when the location may in fact be variant between individuals. In any given subject, most of these seeds may be part of the network of interest (within-network seeds), while some may not (outside-network seeds). The majority of the signal arises from the within-network seeds, since they may all generally have the same connectivity profile. The outside-network seeds may be randomly distributed across other networks, and may thus act as background noise. As a result, there may be a very low probability of imprecision due to inter-individual variability in seed localization.

An alternative method that has been used to determine neurostimulation targets includes use of a whole-brain classifier. However, this method typically requires computation of connectivity with tens of thousands (or hundreds of thousands) of distinct brain voxels followed by dimensionality reduction. In contrast, the targeting method described herein generally comprises computation of connectivity with a limited number of sub-parcels, which is less computationally intensive compared to the whole-brain classifier method. The number of sub-parcels may range from 2 to 50 sub-parcels, including all values and sub-ranges therein. For example, the number of sub-parcels may be 2, 5, 10, 15, 20, 25, 30, 35, 40, 45, or 50 sub-parcels. In one variation, the method includes computation of connectivity with 50 sub-parcels. The number of sub-parcels may also be larger than 50 sub-parcels, in variations where more fine-grained parcellation or parcellation of a very large region of interest is desirable.

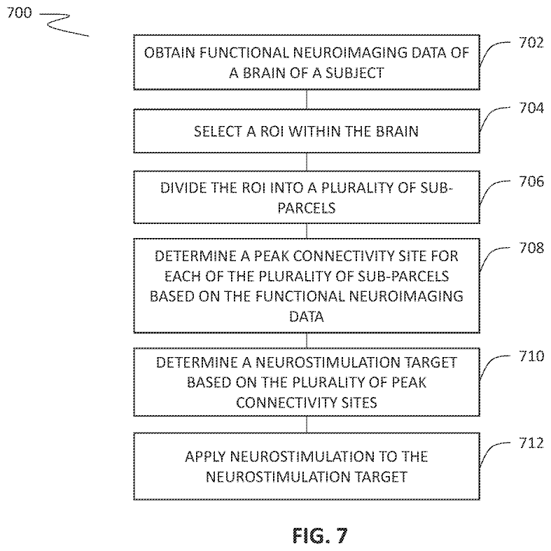

The methods described herein may be able to determine target neurostimulation sites based on functional neuroimaging data of the subject without requiring that the imaging data be recognizable by a machine learning algorithm. In some variations, the method may include the algorithm outlined in FIG. 7. Referring to FIG. 7, the algorithm (700) may include the step of obtaining functional neuroimaging data of a brain of the subject (702), where the functional neuroimaging data describes neuronal activation within the brain. The functional neuroimaging data may be in one of the following modalities: MRI, fMRI, diffuse optical imaging (“DOI”), computer-aided tomography (“CAT”), event-related optical signal (“EROS”) imaging, Magnetoencephalography (“MEG”), positron emission tomography (“PET”) imaging, single-photon emission computerized tomography (“SPECT”) imaging, or electroencephalography (“EEG”). The functional neuroimaging data may relate to the entire brain, portions of the brain, or to pre-defined regions within the brain. In certain variations, the functional neuroimaging data may comprise data sufficient for a four-dimensional (“4D”) reconstruction of neuronal activity within the imaged brain region(s) or for a three-dimensional (“3D”) reconstruction. The 4D reconstruction may be characterized by voxel resolution, scanning frequency or sampling rate, and scanning duration. The voxel resolution may typically range from about 0.5 mm to about 8.0 mm, including all values and sub-ranges therein, although finer voxel resolution (e.g., less than about 0.5mm) may be used if supported by the imaging method employed. Sampling rate (that is, temporal resolution or the reciprocal of scanning frequency) may typically range from less than about 1.0 ms to about 3.0 seconds, including all values and sub-ranges therein, although lower or higher sampling rates could be used if supported by the imaging method employed. Scanning duration may typically range from about 3.0 seconds to about 3 hours, although in some instances durations outside this range may be used.

fIVIRI is an imaging modality that may be used to observe functional connectivity within the brain. However, this technology may be cumbersome to use due to its size, weight, and cost. Further, a fMRI instrument employs radiation to image the brain which carries safety risks for both patients and clinicians. Given this, it would be beneficial to have a method and system for measuring functional connectivity that is safe, cost-effective, and easy to use. In some variations, functional near infrared spectroscopy (fNIRS) may be used. fNIRS is an imaging modality that employs light to measure functional connectivity in the brain. This modality is generally cost effective, light, and easy to use. Although fNIRS may capture signals effectively, this modality is not able to measure depth. Without this capacity, a clinician is unable to administer treatment to a defined target. To circumvent this issue, ultrasound, as well as doppler variations, may be used in conjunction with fNIRS to assist a clinician in delivering treatment to the correct location. In yet further variations, doppler ultrasound may be used to measure functional connectivity.

Next, the algorithm (700) may include selecting a region of interest (“ROI”) within the subject's brain (704). The ROI may be an anatomical region within the brain associated with mood, such as the limbic system. For example, the region of interest may be the amygdala, the hippocampus, or the dorsomedial thalamus. It is understood that other regions of interest could be used. In some variations, the ROI may include or be associated with a brain network. For example, the brain network may be the dorsal attention network (DAN) or the anxiosomatic circuit, as previously described.

The DAN, also known anatomically as the dorsal frontoparietal network (“D-FPN”), is a large-scale brain network that is primarily composed of the intraparietal sulcus (IPS) and frontal eye fields (FEF). It may also include the middle temporal region (MT+), superior parietal lobule (SPL), supplementary eye field (SEF), and ventral premotor cortex. Reduced connectivity within the dorsal and ventral attention networks has been linked to higher levels of attention deficit hyperactivity disorder symptoms. Reduced connectivity between the DAN and the frontoparietal network may be associated with major depressive disorder. On the other hand, overactivation of the DAN has been observed in patients with schizophrenia. The anxiosomatic circuit is a network of brain regions defined by connectivity to sites that, when stimulated with TMS, may relieve anxious and somatic symptoms in patients with major depression. It generally includes a region within the dorsomedial prefrontal cortex which may be used as a TMS target.

Directly stimulating a brain network may not be practicable for a number of reasons, such as neuroanatomical diffuseness and difficult access due anatomical depth and thus distance from the brain surface. For example, the DAN is widespread and generally covers disparate portions of the brain, as the IPS, FEP, MT+, SPL, and SEF, which are not adjacent brain regions. As such, attempting to stimulate most or all of the brain regions comprised in a brain network, in addition to the practical difficulty of how to effectively stimulate a wide area of neural tissue, may create safety and other complications. Therefore, it may be beneficial to locate a neurostimulation target whose activity is more neuroanatomically compact and more easily targeted, and whose stimulation would be expected to robustly modulate the activity of the brain network.

Following step 704, the ROI may be divided into a plurality of sub-parcels (706), and a peak connectivity site for each of the plurality of sub-parcels determined based on the functional neuroimaging data (708). In some instances, the peak connectivity site for each sub-parcel may be characterized by a pattern of neuronal activity having a high degree of correlation or anti-correlation of neuronal activity in corresponding areas of the prefrontal cortex. Each sub-parcel of the ROI may be referred to herein as a “seed”.

As previously described, the ROI may be divided (“parcellated”) into between 2 and 100 sub-parcels or seeds. Each seed may be divided in accordance with a predetermined voxel volume, or in accordance with a published parcellation map or a brain atlas. Examples of a published parcellation map or brain atlas include a Schaefer parcellation map, Mindboggle 101, the Consensual Atlas of Resting-State Networks (CAREN), MICCAI 2012, the Brainnetome Atlas Parcellation, the Harvard Oxford Cortical/Subcortical Atlas, AICHA (Atlas of Intrinsic Connectivity of Homotopic Areas), the Hammersmith Atlas, the Yeo Functional Parcellation, and the JuBrain/Juelich Atlas.

The peak connectivity site may be within the ROI or outside of the ROI. The peak connectivity site for a given seed may be determined based on a degree of synchrony of neuronal activity as determined by functional neuroimaging of a given sub-parcel. The peak connectivity site may be selected from within a candidate target region in the brain. In some variations, the candidate brain region may be overlapping, partially overlapping, or non-overlapping with the given sub-parcel. In other variations, the candidate brain region may be overlapping, partially overlapping or non-overlapping with the ROI as a whole. The candidate target region may be a brain region that is known, based on prior functional imaging, electrophysiological, or anatomical studies, to exhibit neural connectivity with the ROI, and is also anatomical accessible to neurostimulation. A brain region that may be anatomically accessible to neurostimulation may be characterized by being a relatively superficial region of the brain, for example, a portion of the cortex that is within a threshold depth from the skull. When the ROI includes or is associated with the DAN, the candidate target region may be the dorsolateral prefrontal cortex. When the ROI is the anxiosomatic circuit, the candidate target region may be the entire prefrontal cortex.

In one variation, the peak connectivity site may be determined by determining a degree of correlation between a sub-component of a seed and other sub-components within the seed; and determining a degree of correlation between the sub-component of the seed and sub-components within the other seeds. In another variation, a portion of the candidate target region demonstrating the highest degree of correlation or anti-correlation is designated as the peak connectivity site for the sub-parcel. As such, the above analysis for 30 sub-parcels of an ROI may result in the designation of 30 peak connectivity sites, one peak connectivity site for each of the sub-parcels. Each peak connectivity site may be characterized by a set of brain anatomy coordinates of the subject.

In some variations, the reliability of the designation of respective peak correlation site for each seed may be assessed. Optionally, the reliability may be assessed by dividing the functional neuroimaging data into two or more data subsets, repeating the designation of the peak correlation sites with each data subset, and comparing peak connectivity sites designated based on each data subset. By way of example, a coordinate C₁ for a peak correlation site may be determined for a sub-parcel SP₁ based on each of data sets DS1 and DS2, thus creating coordinate C_{1,1}based on data set DS1 and coordinate C_{1,2} based on data set DS2. The process may be repeated for each of N sub-parcels SP_{N} to generate N coordinates C_{N,1} based on data set DS1 and other N coordinates C_{N,2} based on data set DS2. The distance between each pair of coordinates C_{N,1} and C_{N,2} may then be calculated, and the peak connectivity sites determined to be reliable if the average distance is below a predetermined threshold. Additionally or alternatively, the reliability of the designation of the peak correlation sites may be assessed through a Monte Carlo Simulation.

Referring to FIG. 7, once the peak connectivity sites (PCSs) are designated and validated for reliability, the PCS coordinates may be used to determine a neurostimulation target for the subject (710). In some instances, for example, if the PCS coordinates cluster in a similar area, the outliers may be dropped and the location of the cluster may be designated as the neurostimulation target. In other instances, a count of PC Ss is calculated for each portion of the candidate target region, and one or more sub-regions within the candidate target region having a count above a predetermined threshold may be designated as being included within the neurostimulation target. Alternatively, the neurostimulation target may consist of one sub-region within the candidate target region. Following step (710), the method may include applying neurostimulation to the determined neurostimulation target. The neurostimulation may be applied as transcranial magnetic stimulation. In one variation, the neurostimulation is applied according to the SAINT protocol described in further detail below.

The different types of anxiety disorders may have different neurostimulation targets for treatment. Using the methods described herein, the neurostimulation target for treating GAD may be within the right dorsolateral prefrontal cortex (R-DLPFC). The target within the R-DLPFC may be defined by anti-correlation with the amygdala. Alternatively, the target within the R-DLPFC may be defined by correlation with the anxiosomatic circuit. In some variations, the target that is anti-correlated with the amygdala may be used for anxiety-related disorders such as panic disorders or phobias, and the target correlated with the anxiosomatic target may be used for generalized anxiety and/or anxiety characterized by anxious or somatic symptoms.

Neurostimulation targets may also vary depending on the symptoms experienced by the subject, for example, symptoms experienced in addition to anxiety. In some instances, the neurostimulation target may be the anxiosomatic circuit when the subject is experiencing symptoms of insomnia and sexual dysfunction.

### Delivery of Neurostimulation

Magnetic stimulation may be applied to the neurostimulation target in various ways. In one variation, the magnetic stimulation may be aTBS (such as aiTBS or acTBS) delivered transcranially according to the SAINT (Stanford Accelerated Intelligent Neuromodulation Therapy) protocol, which has been associated with improvement in clinical symptoms in less than one week. The SAINT protocol may include applying aTBS pulses for multiple sessions per day, for several days. In one variation, the SAINT protocol may include the delivery of neurostimulation to a brain target for five days. More specifically, the neurostimulation is delivered to the brain target for 10 sessions a day, with each session lasting 10 minutes, and an intersession interval (the interval between sessions) of 50 minutes. In one variation, the protocol may include the delivery of neurostimulation to a brain target for one day, after which symptoms and/or biosignals associated with the anxiety-related disorder are evaluated and treatment is continued the following day(s) if symptoms and/or biosignals have not met a criterion predictive of response or remission of the disorder.

The stimulation frequency of the aTBS pulses may range from about 20 Hz to about 70 Hz, including all values and sub-ranges therein. For example, the stimulation frequency may be about 20 Hz, about 25 Hz, about 30 Hz, about 35 Hz, about 40 Hz, about 45 Hz, about 50 Hz, about 55 Hz, about 60 Hz, about 65 Hz, or about 70 Hz. When iTBS is used, the burst frequency (that is, the reciprocal of the period of bursting, for example if a burst occurs every 200 ms the burst frequency is 5 Hz) of the aTBS pulses may range from about 3 Hz to about 7 Hz, including all values and sub-ranges therein. For example, the burst frequency may be about 3 Hz, about 4 Hz, about 5 Hz, about 6 Hz, or about 7 Hz. The number of pulse trains may range from 2 to 10. For example, the number of pulse trains may be 2, 3, 4, 5, 6, 7, 8, 9, or 10.

The subject may undergo multiple treatment sessions per day. In some variations, the number of treatment sessions per day may range from 2 sessions to 40 sessions. For example, the number of treatment sessions may be 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40. The number of sessions for iTBS may range from 3 to 15 sessions per day. When cTBS is employed, the number of sessions may range from 10-40 sessions per day. The sessions may be performed on consecutive or non-consecutive days.

Additionally, the duration of the intersession interval may vary and range from about 25 minutes to about 120 minutes, including all values and sub-ranges therein. For example, the intersession interval may be about 25 minutes, about 30 minutes, about 35 minutes, about 40 minutes, about 45 minutes, about 50 minutes, about 55 minutes, about 60 minutes, about 65 minutes, about 70 minutes, about 75 minutes, about 80 minutes, about 85 minutes, about 90 minutes, about 95 minutes, about 100 minutes, about 105 minutes, about 110 minutes, about 115 minutes, or about 120 minutes.

In variations using iTBS, the pulse parameters may include 3-pulse trains with 50 Hz pulses at a burst frequency of 5 Hz for 2 second trains, with trains every 10 seconds for 10 minute sessions (1,800 total pulses per session). In some variations, the iTBS schedule may include conducting 10 sessions per day with 50 minute intersession intervals for 5 consecutive days (18,000 pulses per day, and 90,000 total pulses).

When cTBS is used, pulse trains may range from about 4 seconds to about 45 seconds, including all values and sub-ranges therein. For example, the pulse train may be about 4 seconds, about 5 seconds, about 10 seconds, about 15 seconds, about 20 seconds, about 25 seconds, about 30 seconds, about 35 seconds, about 40 seconds, or about 45 seconds. In one cTBS variation, the pulse parameters may include 3-pulse trains with 50 Hz pulses at a burst frequency of 5 Hz for 40 second sessions (600 total pulses per session). In another variation, the cTBS pulse parameters may include 3-pulse trains with 30 Hz pulses at a burst frequency of 6 Hz for 44 second sessions (800 total pulses per session). In many cTBS variations, 30 sessions may be applied per day with 15-minute intersession intervals for 5 consecutive days (18,000 pulses per day, 90,000 total pulses).

It is understood that the pulse parameters and treatment schedules for applying aTBS may be varied. For example, the number of pulses or frequency of sessions may be increased or decreased depending on the refractoriness of the anxiety and/or the severity of anxiety. In some variations, the number of pulses per session may range from about 600 to about 2,400 depending on the type of aTBS applied.

### Treatment of Generalized Anxiety Disorder (GAD)

GAD is typically treated with a combination of psychotherapy and medications such as antidepressants, buspirone, or benzodiazepines. The approaches described herein use non-invasive electromagnetic stimulation, e.g., transcranial magnetic stimulation, to treat GAD. The approaches described herein may also be used with, e.g., transcranial pulsatile stimulation, transcranial focused ultrasound stimulation, or electrical stimulation delivered using permanently or temporarily implanted epidural, subdural, deep brain, intracalvarial, or other electrodes located at or near the stimulation target. Details of a study to examine the effectiveness of aTBS on GAD symptoms is provided in Example 1. The neurostimulation will be delivered for 10 sessions a day for five consecutive days, for a total dose of 18,000 pulses per day. Each session will be 10 minutes each, and the intersession interval will be set at 50 minutes.

The study will also examine the effect of aTBS when the neurostimulation is applied to two personalized targets in the right dorsolateral prefrontal cortex (R-DLPFC) of each subject. The first target (Target 1) will have a maximum resting state fMRI anti-correlation with the amygdala. The second target (Target 2) will have a maximum resting state fMRI correlation with the anxiosomatic circuit. Functional connectivity and information relating to one or more symptoms experienced by the subject in addition to anxiety, and/or one or more clinical factors will also be included to help determine the effect of aTBS on the two personalized targets.

Transcranial magnetic stimulation (TMS) may be applied to the personalized targets, as mentioned above. TMS is a neuropsychiatric therapy where electric pulses are administered to the brain at a region whose activation will cause a desired downstream effect. However, in some instances, this technology may be cumbersome to use due to the amount of energy that is needed to power high voltage instruments. Further, treating an individual with electrical pulses may pose a safety risk as clinicians and patients are exposed to dangers such as dermal burns and electrocution. Even further, TMS does not have the capacity to stimulate deep brain structures. Given this, it may be beneficial to administer neurostimulation using safer modalities. In some variations, focused ultrasound (fUS) may be employed to administer the neurostimulation therapy. In contrast to electricity, fUS uses sound to activate desired regions of the brain. In addition to being safer than TMS, fUS has the capacity to stimulate deep brain structures, which may allow clinicians to bypass stimulating cortical target regions. Further, fUS may also be used at multiple target regions, or in conjunction with TMS, to provide multi-targeted neurostimulation therapy.

### Concurrent Treatment of Disorders

Currently available stimulation treatment protocols for neurological and psychiatric disorders generally include the application of therapy to a single treatment target during a treatment session or during a course of treatment (which may include multiple treatment sessions). In some instances, these protocols may prevent clinicians from treating two or more disorders concurrently, may prevent the design of combination therapies (e.g., the application of therapy at multiple targets for the treatment of a single condition), and/or may fail to prevent side-effects of stimulation. Accordingly, in addition to identifying personalized targets for treatment of GAD, some variations of the method may use functional connectivity to identify target areas that may concurrently treat two or more disorders. The two or more disorders may include psychiatric disorders such as, but not limited to, anxiety disorders, anxiety-related disorders, depression, pain disorders, substance use disorders, bipolar disorder, and schizophrenia. The anxiety and anxiety-related disorders may be generalized anxiety disorder, panic disorder, post-traumatic stress disorder (PTSD), obsessive-compulsive disorder (OCD), or phobias such as social phobia or agoraphobia. The two or more disorders may also be neurological disorders such as, but not limited to, Parkinson's disease, essential tremor, stroke, epilepsy, traumatic brain injury, migraine headache, cluster headache, and chronic pain. In some variations of the method, the two or more disorders that are treated by stimulation therapy may be non-neuropsychiatric disorders, and include without limitation, inflammatory bowel disease, lupus, rheumatoid arthritis. The stimulation concurrently applied to two or more target regions may include transcranial magnetic stimulation, focused ultrasound, or both.

For example, if a patient presents with depression and anxiety (e.g., GAD), potential targets may be identified by locating target regions where neurostimulation results in the desired effect in reference regions of interest. The standard neurostimulation treatment for depression may generally involve the application of neurostimulation to cortical regions of the brain, which when stimulated, decreases activation in the subgenual anterior cingulate cortex, whereas the standard treatment for anxiety (e.g., GAD) may generally involve the application of neurostimulation to cortical regions of the brain, which when stimulated, decreases activation in the amygdala. Given this, both anxiety (e.g., GAD) and depression may be treated concurrently by identifying a single target region of the brain cortex that is functionally connected to both the subgenual anterior cingulate cortex and amygdala such that application of stimulation to the single target region will decrease activation in both the subgenual anterior cingulate cortex and the amygdala. Functional connectivity may be measured using imaging modalities, such as fNIRS, fMRI, and/or doppler ultrasound.

fMRI is an imaging modality often used to observe functional connectivity within the brain. However, this technology may be cumbersome to use due to its size, weight, and cost. Further, a fMRI instrument employs radiation to image the brain which carries safety risks for both patients and clinicians. Given this, it would be beneficial to have a method and system for measuring functional connectivity that is safe, cost-effective, and easy to use. In some variations, functional near infrared spectroscopy (fNIRS) may be used. fNIRS is an imaging modality that employs light to measure functional connectivity in the brain. This modality is generally cost effective, light, and easy to use. Although fNIRS may capture signals effectively, this modality is not able to measure depth. Without this capacity, a clinician is unable to administer treatment to a defined target. To circumvent this issue, ultrasound, as well as doppler variations, may be used in conjunction with fNIRS to assist a clinician in delivering treatment to the correct location. In yet further variations, doppler ultrasound may be used to measure functional connectivity.

In some variations, the concurrent treatment of neuropsychiatric disorders may reduce or prevent the occurrence of side-effects from neurostimulation therapy. Standard neurostimulation protocols may not identify treatment targets that are likely to cause the presentation of a side-effect when stimulated. As previously described herein, current neurostimulation treatment protocols for neurological and psychiatric disorders generally employ functional connectivity networks to identify treatment targets. To identify such targets, functional connectivity between two or more regions, often a cortical region and a deep brain region, may be measured, and then treatment applied based on the degree of synchrony or asynchrony between levels of activation in each respective region. In view of this, treatment targets that when stimulated are unlikely to produce a side-effect may be located by identifying those targets that are not functionally connected or that are maximally non-correlated (e.g., non-synchronous) to reference regions of interest whose activation or deactivation may cause a side-effect. For example, neurostimulation therapy for the treatment of depression may be applied to target regions in the dorsolateral prefrontal cortex (DLPFC) for which there may be anti-correlation to the subgenual anterior cingulate cortex. However, given that activation of specific regions of the amygdala may cause symptoms of anxiety (a side effect in this instance), to treat a patient that has presented with depression, it may be beneficial to stimulate a target area that may decrease activation in the subgenual anterior cingulate cortex, but which may not cause downstream activation within regions of the amygdala, as this may cause or exacerbate symptoms of anxiety. Such target areas may be identified by identifying regions that are both maximally anti-correlated to the subgenual anterior cingulate cortex and maximally non-correlated to the amygdala. In general, there may be anti-correlation when an increase in activation in the target region, decreases activation in the region of interest and vice versa. There may be non-correlation when an increase or decrease in activation in the target region does not change activation in the region of interest. It may be helpful to prevent the presentation of anxiety as a side-effect as this symptom may cause an individual to ruminate on their condition, which could lead to relapse.

In other variations, the concurrent treatment of one or more neuropsychiatric disorders may include increasing the number of target regions to which neurostimulation is applied. For example, when the SAINT protocol is used, as described herein, it may be useful to apply neurostimulation to two or more target regions concurrently or alternatively during treatment sessions. In some variations, the concurrent treatment may shorten the duration of the total treatment protocol. For example, if a patient presents with both depression and anxiety, both conditions may be treated at the same time. In other variations, the concurrent treatment may decrease the number of treatment sessions for a patient.

In further variations, the concurrent treatment of neuropsychiatric disorders may include determining target regions that when stimulated concurrently do not interfere with activation of each other. As previously described, standard protocols generally measure functional connectivity between target and reference regions to identify potential target regions. However, they do not measure the functional connectivity between target regions to predict target viability. Given this, to ensure that stimulation at a primary target region does not change activation in a secondary target region, functional connectivity measures may be used to determine whether stimulation may be applied to respective target regions, concurrently or alternatively. For example, if stimulation at target region A increases or decreases activation at target region B, these target regions should not be stimulated concurrently. However, if stimulation at target region A is functionally non-correlated to target region C, these targets may be stimulated concurrently.

Machine learning algorithms for analyzing functional neuroimaging data are generally trained on data obtained from healthy patients, which are more readily available than data from subjects with a given brain pathology of interest. However, there is a concern that algorithms trained from healthy brains are sub-optimal for analyzing data obtained from patients with brain pathologies. Thus, variations of the methods described herein may be configured to determine targets regions for delivering neurostimulation to a patient having a brain pathology based on the patient's own functional neuroimaging data, thus personalizing identification of the neurostimulation targets. In some variations, as further illustrated in Example 2, the machine learning model may be used in a method for determining target regions in a patient having more than one psychiatric or neurological disorder. In other variations, as further illustrated in Example 3, the machine learning model may be used in a method for determining multiple target regions for application of stimulation to treat a patient having a single neuropsychiatric or neurological disorder.

Transcranial magnetic stimulation (TMS) may be used to concurrently treat disorders, as mentioned above. TMS is a neuropsychiatric therapy where electric pulses are administered to the brain at a region whose activation will cause a desired downstream effect. However, in some instances, this technology may be cumbersome to use due to the amount of energy that is needed to power high voltage instruments. Further, treating an individual with electrical pulses may pose a safety risk as clinicians and patients are exposed to dangers such as dermal burns and electrocution. Even further, TMS does not have the capacity to stimulate deep brain structures. Given this, it may be beneficial to administer neurostimulation using safer modalities. In some variations, focused ultrasound (fUS) may be employed to administer the neurostimulation therapy. In contrast to electricity, fUS uses sound to activate desired regions of the brain. In addition to being safer than TMS, fUS has the capacity to stimulate deep brain structures, which may allow clinicians to bypass stimulating cortical target regions. Further, fUS may also be used at multiple target regions, or in conjunction with TMS, to provide multi-targeted neurostimulation therapy.

### Systems

Systems for treating anxiety disorders are also disclosed herein. The systems may include a transcranial magnetic stimulation coil configured to apply accelerated Theta-Burst Stimulation (aTBS) to the neurostimulation target. In one variation, the aTBS may be accelerated intermittent Theta-Burst Stimulation (aiTBS). In another variation, the aTBS may be accelerated continuous Theta-Burst Stimulation (acTBS). aTBS may be delivered to treat various types of anxiety, including but not limited to, generalized anxiety disorder, panic disorder, and phobias such as social phobia or agoraphobia.

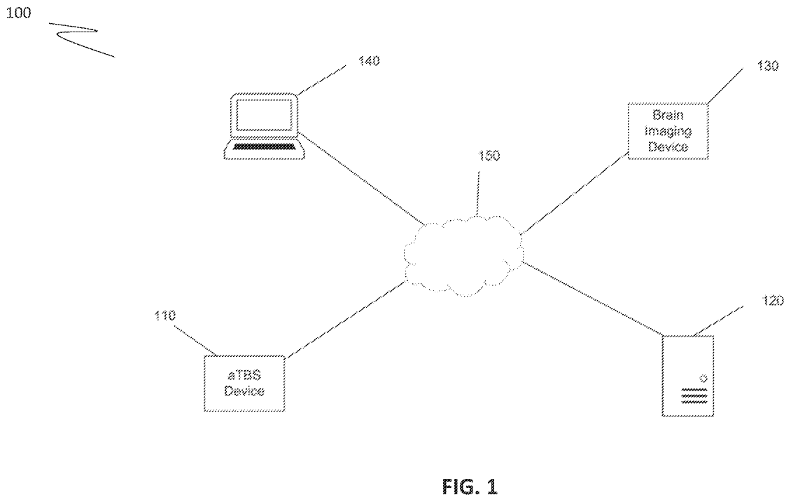

In some variations, the aTBS systems may be configured as shown in FIG. 1. Referring to FIG. 1, aTBS system (100) may include an aTBS device (110). The aTBS devices (110) are generally TMS coils that are capable of delivering magnetic pulses with the frequencies, intensities, and durations required by aTBS. Exemplary aTBS devices may include without limitation, a Magventure X100 produced by MagVenture of Farum, Denmark, Magstim coils produced by The Magstim Company Limited of Whitland, United Kingdom, Neurosoft coils produced by Neurosoft of Utrecht, Netherlands, the Brainsway H7-deep-TMS system, or the H1 Coil TMS device, both of which are produced by Brainsway Ltd. of Jerusalem, Israel. In one variation, aTBS systems may include more than one aTBS device in order to better target different regions of the brain. Neuronavigation systems may be used to place the aTBS device relative to the aTBS target. For example, without limitation, the Localite TMS Navigator produced by Localite GmbH of Sankt Augustin, Germany, visor2 systems produced by ANT Neuro of the Netherlands, or the BrainSite TMS Navigation System produced by Rogue Solutions Ltd. of Cardiff, Wales may be employed to display the aTBS target and place the aTBS device. Other exemplary aTBS devices may include without limitation, aTBS devices that are integrated or combined with a neuronavigation system, such as the Nexstim NBT 2 system produced by Nexstim Oyj of Finland.

The aTBS system (100) may further include an aTBS computing system (120) and a brain imaging device (130). aTBS computing systems may be implemented as one or more computing devices capable of processing brain image data, information relating to patient symptoms and/or clinical factors, and generating personalized aTBS targets. Brain imaging devices may obtain structural imaging data, functional imaging data, and/or resting state imaging data. For example, the brain imaging devices may be magnetic resonance imaging (MM) machines or functional MM (fMRI) machines. Functional imaging data may be obtained by scanning a patient using an fMRI scanner or other brain imaging device, while the patient is performing specific tasks and/or is provided specific stimuli. Resting state imaging data may be obtained by scanning a patient using an fMRI scanner, or other brain imaging device while the patient is not performing any task.

Additionally, aTBS system (100) may include an interface device (140). Interface devices may be, but are not limited to, computers, smart-phones, tablet computers, and smart-watches. In some variations, interface devices may be used to interface with brain imaging devices, aTBS computing systems, and/or aTBS devices. In one variation, aTBS computing systems and interface devices may be implemented using the same physical device. Referring to FIG. 1, for example, aTBS system (100) includes a network (150) connecting aTBS device (110), aTBS computing system (120), and interface device (130).

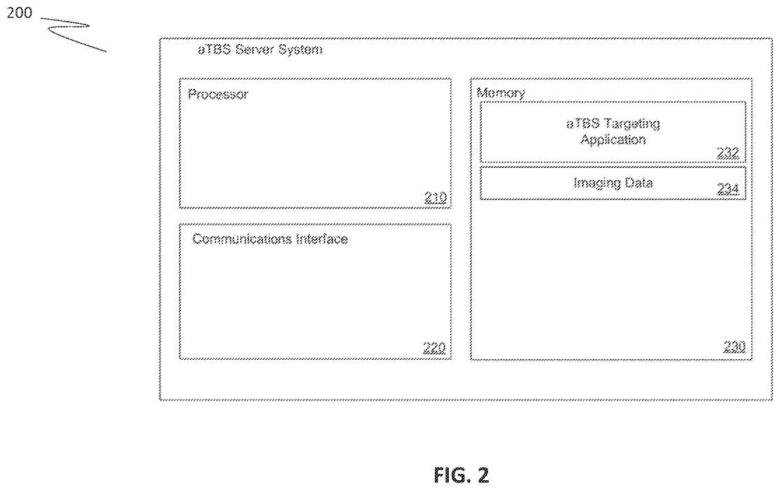

The aTBS computing systems may be configured as shown in FIG. 2. In FIG. 2, aTBS computing system (200) may include a processor (210) in communication with a communications interface (220) and a memory (230). In some variations, aTBS computing systems may comprise multiple processors, multiple memories, and/or multiple communications interfaces. In other variations, components of aTBS computing systems may be distributed across multiple hardware platforms. Processor (210) may be any type of computational processing unit, including, but not limited to, microprocessors, central processing units, graphical processing units, and parallel processing engines. Communications interface (220) may be utilized to transmit and receive data from other aTBS computing systems, brain imaging devices, aTBS devices, and/or interface devices. Communications interfaces may also include multiple ports and/or communications technologies in order to communicate with various devices as appropriate. Memory (230) may be volatile and/or non-volatile memory. For example, memory (230) may comprise random access memory, read-only memory, hard disk drives, solid-state drives, and flash memory. Memory (230) may store a variety of data, including, but not limited to, an aTBS targeting application (232) and imaging data (234). In some variations, the aTBS targeting application and/or the imaging data may be received via the communications interface. Processor (210) may be directed by the aTBS targeting application to perform a variety of aTBS processes, including, but not limited to, processing imaging data and generating aTBS targets.

Furthermore, aTBS computing systems can be implemented on multiple servers within at least one computing system. For example, aTBS computing systems may be implemented on various remote “cloud” computing systems applications, or partially on a “cloud” computing system and partially on a local computing device integrated into a medical device. Other exemplary computing systems include without limitation, personal computers, servers, clusters of computing devices, and/or computing devices incorporated into medical devices.

In other variations, the systems may include a transcranial magnetic stimulation coil, a data module configured to receive data comprising functional neuroimaging data of a brain of a subject, where the functional neuroimaging data describes neuronal activation within the brain, and a targeting module comprising a memory storing a set of instructions. The targeting module may include one or more processors responsive to the set of instructions that are configured to determine a neurostimulation target in the right hemisphere of the brain based on one or symptoms experienced by the subject in addition to anxiety, and functional neuroimaging data.

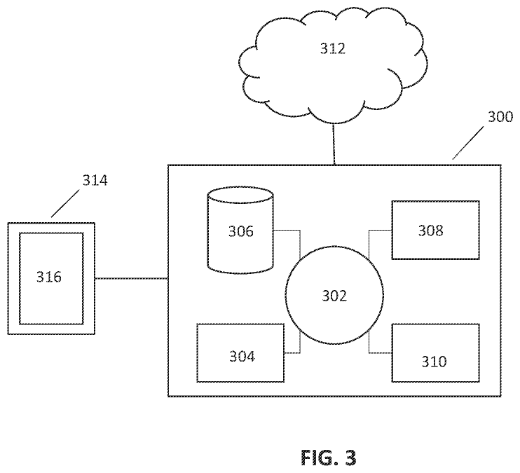

For example, FIG. 3 shows a computer system (300) that may be programmed to obtain functional neuroimaging data of a brain of a subject and determine a neurostimulation target based on functional neuroimaging data. The computer system (300) may be an electronic device of a user or a computer system that is remotely located with respect to the electronic device. The electronic device may be a mobile electronic device.

The computer system (300) includes a central processing unit (CPU, also “processor” and “computer processor” herein) (302), which can be a single core or multi core processor, or a plurality of processors for parallel processing. The computer system (300) also includes memory or memory location (304) (e.g., random-access memory, read-only memory, flash memory), electronic storage unit (306) (e.g., hard disk), communication interface (308) (e.g., network adapter) for communicating with one or more other systems, and peripheral devices (310), such as cache, other memory, data storage and/or electronic display adapters. The memory (304), storage unit (306), interface (308) and peripheral devices (310) are in communication with the CPU (302) through a communication bus (solid lines), such as a motherboard. The storage unit (306) can be a data storage unit (or data repository) for storing data. The computer system (300) can be operatively coupled to a computer network (“network”) (312) with the aid of the communication interface (308). The communication interface may be wired or wireless. The network (312) may be the Internet, an internet and/or extranet, or an intranet and/or extranet that is in communication with the Internet. The network (312) in some cases is a telecommunication and/or data network. The network (312) may also include one or more computer servers, which may enable distributed computing, such as cloud computing. The network (312), in some cases with the aid of the computer system (300), may implement a peer-to-peer network, which may enable devices coupled to the computer system (300) to behave as a client or a server.

The CPU (302) may execute a sequence of machine-readable instructions, which may be embodied in a program or software. The instructions may be stored in a memory location, such as memory (304). The instructions may be directed to the CPU (302), which may subsequently program or otherwise configure the CPU (302) to implement the methods described herein. Examples of operations performed by the CPU (302) may include fetch, decode, execute, and writeback.

The CPU (302) may be part of a circuit, such as an integrated circuit. One or more other components of the system (300) may be included in the circuit. In some cases, the circuit may be an application specific integrated circuit (ASIC).

The storage unit (306) may store files, such as drivers, libraries and saved programs. The storage unit (306) may store user data, e.g., user preferences and user programs. The computer system (300) in some cases may include one or more additional data storage units that are external to the computer system (300), such as located on a remote server that is in communication with the computer system (300) through an intranet or the Internet.

The computer system (300) may communicate with one or more remote computer systems through the network (312). For instance, the computer system (300) may communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system (30) via the network (312).

The methods as described herein may be implemented by way of machine (e.g., computer processor) executable code stored on an electronic storage location of the computer system (300), such as, for example, on the memory (304) or electronic storage unit (306). The machine executable or machine-readable code can be provided in the form of software. During use, the code may be executed by the processor (302). In some cases, the code may be retrieved from the storage unit (306) and stored on the memory (304) for ready access by the processor (302). The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system (300), may be embodied in programming. Various aspects of the technology may be thought of as “products” or “articles of manufacture” typically in the form of machine (or processor) executable code and/or associated data that may be carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks, and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media may include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system (300) can include or be in communication with an electronic display (314) that may comprise a user interface (UI) (316) for providing, for example, a login screen for an administrator to access software programmed to identify a neurostimulation target. Examples of UI's include, without limitation, a graphical user interface (GUI) and web-based user interface.

The methods for treating anxiety disorders and determining personalized targets may be implemented by one or more algorithms by way of software upon execution by the central processing units described herein. The algorithms may use data related to one or more symptoms experienced in addition to anxiety and/or one or more clinical factors from the subject when determining the personalized brain target. The algorithms may deliver neurostimulation to regions within the left or right hemisphere of the brain, but in some instances, it may be useful for the neurostimulation target to reside within the right hemisphere.

The algorithms may be used to treat various types of anxiety. Exemplary anxiety disorders include without limitation, generalized anxiety disorder, panic disorder, and phobias such as social phobia or agoraphobia. In some variations, the algorithms may be used to determine neurostimulation targets for the treatment of generalized anxiety disorder. The algorithms may control an aTBS device such as a TMS coil, which may be configured to deliver aTBS. In one variation, the transcranial magnetic stimulation coil may be configured to apply intermittent Theta-Burst Stimulation (iTBS) to the neurostimulation target. In another variation, the transcranial magnetic stimulation coil is configured to apply continuous Theta-Burst Stimulation (cTBS) to the neurostimulation target.

Based on the fMRI connectivity data, the one or more symptoms in addition to anxiety, and/or one or more clinical factors, the algorithm may determine that the neurostimulation target is a specific part of the dorsolateral prefrontal cortex. In other variations, the algorithm may determine that other cortical regions of the brain may be used as personalized targets. These cortical regions include without limitation, the anterior dorsolateral prefrontal cortex, the dorsal anterior cingulate cortex, the inferior temporal cortex, the lateral parietal cortex, the middle cingulate cortex, the posterior cingulate cortex, the posterior dorsolateral prefrontal cortex, the posterior occipital cortex, the subgenual anterior cingulate cortex, and the ventrolateral prefrontal cortex. In some instances, the region of the brain that is functionally connected to a deep brain structure is the intraparietal sulcus, the pre/post central gyms, the superior parietal lobule, the superior temporal gyms, or the temporal-parietal junction.

As previously mentioned, the algorithm may use a combination of fMRI data and one or more symptoms experienced in addition to anxiety to help determine the personalized target. The one or more symptoms may be depression, panic, fear, fatigue, insomnia, obsessive-compulsiveness, or combinations thereof. Additionally or alternatively, the algorithm may include instructions to obtain and input one or more clinical factors about the subject when determining the neurostimulation target. The one or more clinical factors may be selected from the group consisting of, but not limited to, duration of anxiety, level of anxiety, treatment history for an anxiety disorder, family history of an anxiety disorder, family history of a depressive disorder, post-traumatic stress disorder, an eating disorder, fibromyalgia, demographic information, and combinations thereof.

The systems may include one or more processors operable to run algorithms for determining personalized targets for treating anxiety disorders. The algorithms may be those provided in FIGS. 4-7. Referring to FIG. 4, the one or more processors may run algorithm (400), which first includes the step of compiling a database of targeting approaches for different clusters (groups) of symptoms, clinical factors, and/or diagnoses (402). Next, each targeting approach may be input into the overall targeting decision algorithm (404). Over time, the database may be expanded as studies identify new targeting approaches (406), and more tasks and inventories are added to the battery of options that clinicians may administer (408). Based on the self-report completed by the subject (patient) that includes tasks and inventories that help quantify symptom severity (e.g., depression vs. anxiety severity) (410), the algorithm weights different neurostimulation targets according to the symptoms reported by the subject (412), and may either determine a neurostimulation target based on the most heavily weighted target (414) or determine a sequence of neurostimulation targets based on weighting of different targets (416).

Alternatively, referring to FIG. 5, the one or more processors may run algorithm (500), which also includes the step of compiling a database of targeting approaches for different clusters (groups) of symptoms, clinical factors, and/or diagnoses (502). However, a separate decision algorithm may be created for each targeting approach (504). Over time, the database may be expanded as studies identify new targeting approaches (506), and more tasks and inventories are added to the battery of options that clinicians may administer (508). Based on the self-report completed by the subject (patient) that includes tasks and inventories that help quantify symptom severity (e.g., depression vs. anxiety severity) (510), the algorithm may determine a neurostimulation target based on the symptoms reported by the subject (patient).

In some variations, as shown in FIG. 6, the one or more processors may run algorithm (600), which includes the step of compiling a database of targeting approaches for different clusters (groups) of symptoms, clinical factors, and/or diagnoses (602). An individualized whole-brain connectivity map may then be generated for each possible cluster of symptoms and/or diagnoses (604). Next, for each brain location, connectivity to the target may be extracted for each possible cluster of symptoms and/or diagnoses (606), and improvement in each cluster of symptoms and/or diagnoses predicted as being proportional to the connectivity value (608). Based on this connectivity value, the clinician may select three options. In the first option (610), the clinician may select a symptom and see a map of identified targets for that symptom. In the second option (612), the clinician may select a target in the brain and see a bar graph (or table) depicting the expected improvement in different symptoms if that area is targeted. With the third option (614), an aTBS device (e.g., a TMS coil) may be moved around the brain using neuronavigation, and expected improvements in different symptoms shown on a display that correlate with the brain target over which the TMS coil is positioned.

In other variations, as shown in FIG. 7, the one or more processors may run an algorithm (700) that includes obtaining functional neuroimaging data of a brain of the subject (702), selecting a region of interest within the brain (704), dividing the region of interest into a plurality of sub-parcels (706), and determining a peak connectivity site for each of the plurality of sub-parcels based at least in part on functional neuroimaging data (708). The neurostimulation target may then be determined based om the peak connectivity sites (710). Once determined, the one or more processors may instruct for neurostimulation to be delivered to the target (712).

The one or more processors may also run an algorithm that further includes selecting a subset of the plurality of peak connectivity sites that cluster within a common neuroanatomical region, and designating at least a portion of the common neuroanatomical region as the neurostimulation target. In another variation, the algorithm may assign for each of a plurality of potential target locations, a degree of connectivity to each of the plurality of sub-parcels, and calculate for each of the plurality of potential target locations, an overall degree of connectivity based on a numerical average of the degree of connectivity to each of the plurality of sub-parcels. Based on the overall degree of connectivity, the algorithm may include selecting one or more potential target locations having an overall degree of connectivity above a predetermined threshold as the neurostimulation target.

The methods for concurrent treatment of disorders may be implemented by one or more algorithms by way of software upon execution by the central processing units described herein. In some variations, the algorithms may be used to concurrently treat a patient having two or more disorders, as further described in Example 2. In other variations, the algorithms may be used to determine multiple target regions for application of stimulation to treat a patient having a single neuropsychiatric or neurological disorder.

### EXAMPLES

The following examples are illustrative only, and should not be construed as limiting the disclosure in any way.

### Example 1

### Feasibility Study to Evaluate the Effect of Neurostimulation of Brain Targets in Subjects with Generalized Anxiety Disorder (GAD)

To help establish whether aTBS delivered via the SAINT protocol may be used to treat subjects with GAD, a feasibility study will be conducted with 30 subjects. The feasibility study will also determine whether certain brain targets are associated with one or more symptoms noted by the subject or a clinician. A follow-up randomized clinical trial may be conducted upon results of the feasibility study showing ≥50% reduction in anxiety symptoms as measured by the Hamilton Anxiety Rating Scale (HAM-A). The symptoms will generally be one or more in addition to anxiety. For example, the clinician may complete a report, or the subject will be asked to complete a questionnaire relating to whether one or more of the following symptoms are being experienced: anhedonia, depression, dysphoria, panic, fear, fatigue, insomnia, obsessive-compulsiveness, restlessness, weight change, cognitive change, mood, tension, somatic sensations such as dizziness or numbness, somatic muscular events such as twitching, cardiovascular symptoms such as tachycardia, respiratory symptoms such as constriction in the chest, gastrointestinal symptoms such as looseness of bowels, genitourinary symptoms such as impotence or loss of libido, autonomic symptoms such as dry mouth, feelings of irritation, feelings of guilt, or other symptoms of depression and/or anxiety. Additionally, the questionnaire will ascertain one or more clinical factors about the subject to determine the neurostimulation target. Such clinical factors may include duration of anxiety, severity of anxiety, treatment history for an anxiety disorder, family history of an anxiety disorder, family history of a depressive disorder, post-traumatic stress disorder, an eating disorder, fibromyalgia, demographic information, history of OCD, history of panic attacks, history of major depression, history of substance abuse, history of comorbid pain, or other correlates or comorbidities of depression and/or anxiety.

All subjects will undergo an initial MRI structural and functional scanning sequence as part of their baseline assessment. These MM scans will be used to generate two individualized algorithm-derived targets for neurostimulation. The two brain targets for evaluation will be in the right hemisphere of the subject's brain, and more specifically, two distinct subregions of the right dorsolateral prefrontal cortex (R-DLPFC). The first subregion of the R-DLPFC (Target 1) will have a maximum resting state fMRI anti-correlation with the amygdala. The second subregion of the R-DLPFC (Target 2) will have a maximum resting state fMRI correlation with the anxiosomatic circuit.

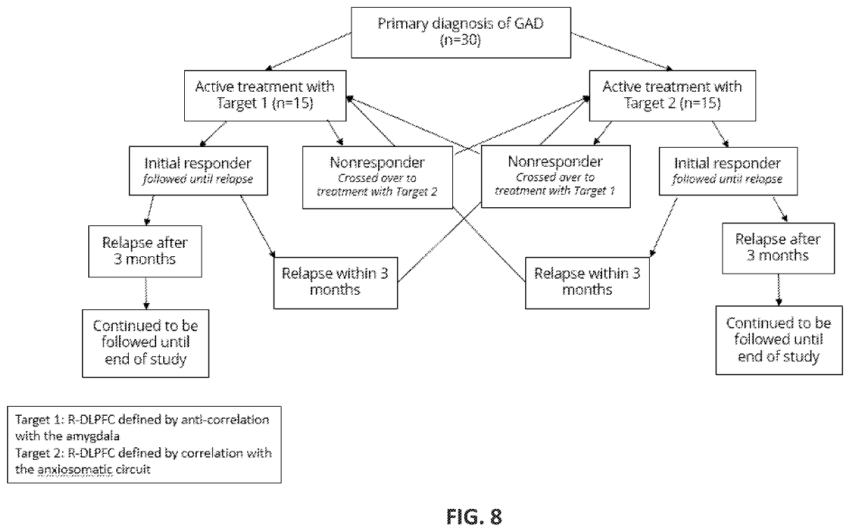

The neurostimulation will be delivered to Target 1 or Target 2 according to the SAINT treatment protocol previously described, that is, for 10 sessions a day for five consecutive days. Each session will be 10 minutes each, and the intersession interval will be set at 50 minutes. Specific parameters of the delivered neurostimulation are provided below in Table 2. Motor threshold determinations will be made at the start of each course of treatment. Referring to FIG. 8, one of two potential targeting strategies will be used in a balanced randomized order of block size of 4 in this crossover design study. Participants not achieving response, defined as at least 50% reduction in the baseline HAM-A score, within 30 days of the final day of treatment, will be crossed over to the other arm of the study and receive 5 days of active SAINT treatment with the alternative target.

Participants that do enter response within 30 days of the acute treatment will have the option to receive another targeting strategy for a second course of acute treatment once they no longer meet responder criteria, as long as relapse occurs within 3 months of their final day of their initial treatment week. Participants who initially remit from the first week of treatment and do not relapse within the 3-month follow-up period will not enter into the cross-over arm of the study but will be followed by weekly self-reports until relapse occurs or the end of the trial, whichever comes first. Participants who enter into the cross-over arm of the study will be followed biweekly by a trained assessment rater for the 3-month follow-up period and then will continue to complete weekly self-reports until relapse occurs or the end of the trial, whichever comes first.

TABLE 2

| | TARGET 1 | | TARGET 2 |
|---|---|---|---|
| | TREATMENT | | TREATMENT |
| Target Identification and | Individualized anatomical | | -same as target 1 treatment except |
| Specificity | landmarks and individual | | for modified target determination |
| | functional connectivity - structural | | algorithm- |
| | MRI + functional MRI + software | | |
| | algorithm to further pinpoint ideal | | |
| | area for stimulation based on | | |
| | individual anatomy and functional | | |
| | connectivity | | |
| Stimulation Dose | 1,800 pulses per session, 18,000 | | -same as target 1 treatment- |
| | pulses per day | | |
| Magnetic Field Intensity | 90% motor threshold with depth | | -same as target 1 treatment- |
| | adjustment | | |
| Pulse Frequency | 50 | Hz | -same as target 1 treatment- |
| Pulses per Burst | 3 | | -same as target 1 treatment- |
| Burst Frequency | 5 | Hz | -same as target 1 treatment- |
| Stimulus Train Duration | 2 | sec | -same as target 1 treatment- |
| Inter-Train Interval | 8 | sec | -same as target 1 treatment- |
| Number of Trains per Session | 60 | | -same as target 1 treatment- |
| Magnetic Pulses per Session | 1800 | | -same as target 1 treatment- |
| Treatment Session Duration | 10 | min | -same as target 1 treatment- |
| Sessions/Day | 10 | | -same as target 1 treatment- |
| Sessions/Week | 50 | | -same as target 1 treatment- |
| Total Sessions | 50 | | -same as target 1 treatment- |
| Total Pulses | 90,000 | | -same as target 1 treatment- |
| Treatment Schedule | 5 | days | -same as target 1 treatment- |

| | | |
|---|---|---|
| Area of Brain Stimulated | Individualized target within R- | Individualized target within R- |
| | DLPFC defined by anticorrelation | DLPFC defined by correlation |
| | with amygdala | with anxiosomatic circuit |

### Example 2

### Concurrent Treatment of Two Disorders

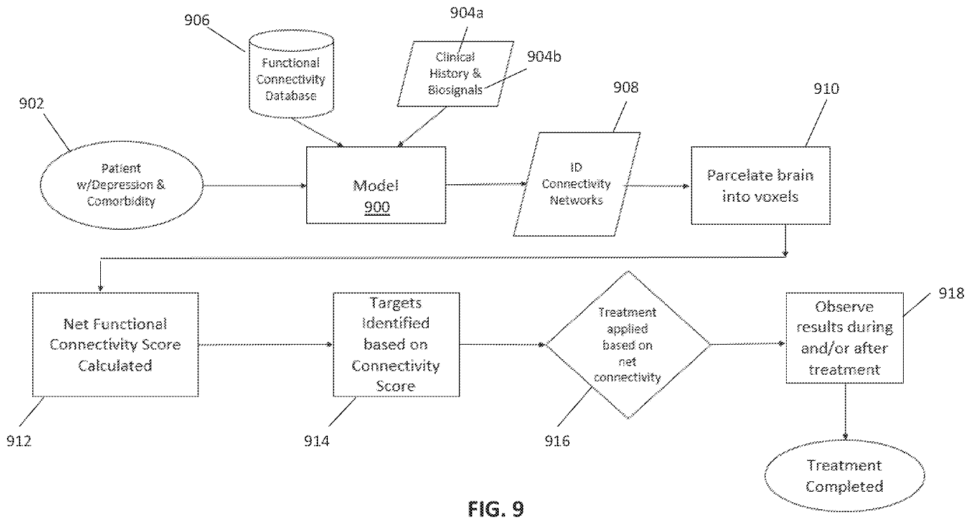

FIG. 9 illustrates exemplary steps of a method including a machine learning model (900) for determining target regions in a patient presenting to the clinic for the treatment of more than one psychiatric or neurological disorder. In this case, the patient (902) may present with depression and another neuropsychiatric disorder such as anxiety (a comorbidity). The machine learning model (900) may receive data for input into the model, such as the patient's clinical history (904a), biosignals (904b), and/or data from a functional connectivity network database (906) that includes data obtained from previously conducted functional connectivity studies. Exemplary biosignals may include without limitation, heart rate, heart rate variability, blood pressure, respiratory rate, skin conductivity, as well as genomic data. The output of the machine learning model may identify one or more connectivity networks of interest (908) based on the functional connectivity of target seeds to the one or more connectivity networks using an imaging modality. The imaging modality may be, e.g., fMRI, fNIRs, ultrasound, or doppler ultrasound. The target seeds may then be parcellated into voxels (910). A net functional connectivity score may then be calculated for each target (912). The net connectivity score may evaluate targets based on their connectedness and disconnectedness to functional connectivity networks of interest. For example, the targeting seeds having the greatest functional connectivity to networks of interest are de-weighted based on their connectivity to networks that, when stimulated, may increase the symptoms of depression, and/or a comorbidity, and/or may cause a side-effect. The machine learning model may then provide one or more optimal target regions based on their net connectivity. For example, net functional connectivity (914) may be calculated by the summation of functional connectivity scores from two or more functional connectivity networks.

The clinician may choose a target from the list of target regions provided. Treatment may then be applied to the chosen target (916). Clinicians may observe stimulation effects in real-time (918), using a modality such as EEG, fNIRS, or doppler ultrasound to determine whether stimulation of a target elicits the desired downstream effects in a brain region of interest.

### Example 3

### Concurrent Treatment Using Multiple Target Regions for a Single Disorder

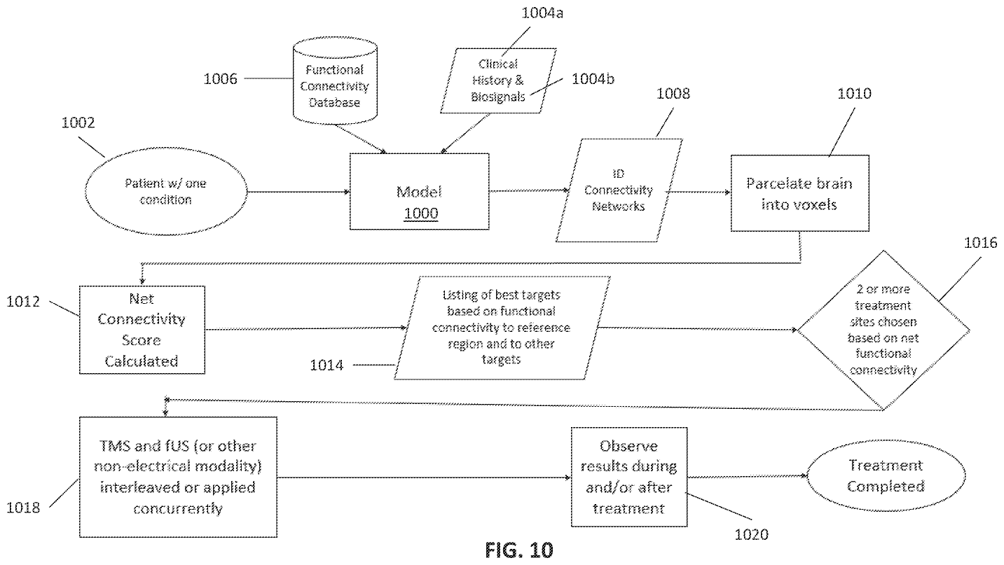

FIG. 10 illustrates exemplary steps of a method including a machine learning model (1000) for determining target regions in a patient (1002) presenting to the clinic for the treatment of a single neuropsychiatric or neurological condition (i.e., disorder). The machine learning model (1000) may receive data for input into the model such as the patient's clinical history (1004a), biosignals (1004b), and/or data from a functional connectivity network database (1006). The output of the machine learning model may identify one or more connectivity networks of interest (1008) based on the functional connectivity of the target seeds to the networks using an imaging modality, such as fMRI, fNIRs, ultrasound, or doppler ultrasound. The target seeds may then be parcellated into voxels (1010). A net functional connectivity score may then be calculated for each target (1012). The net connectivity score may evaluate targets based on their connectedness and disconnectedness to the functional connectivity networks of interest. For example, net functional connectivity may be calculated by the summation of functional connectivity scores from two or more functional connectivity networks where functional correlation may be measured in positive values and functional anti-correlation may be measured in negative values. The machine learning model (1000) may then provide a listing of the optimal targets based on their connectedness to the functional connectivity networks of interest as well as their connectedness to other targets (1014). The clinician may choose two or more targets for treatment from the target list provided (1016).

Treatment of the multiple targets may include the use of TMS (transcranial magnetic stimulation) to treat one target and fUS (focused ultrasound) or another non-electric modality to treat another target (1018). Treatment with multiple modalities may be performed concurrently, sequentially, or simultaneously depending on interactions of the treatment modalities. Clinicians may observe stimulation effects in real-time (1020), using a modality such as EEG, fNIRS, or doppler ultrasound to determine whether stimulation of the targets elicit the desired downstream effect in a brain region(s) of interest.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated.

1. A method for treating an anxiety disorder in a subject comprising:
selecting a region of interest within a brain of the subject;
determining a neurostimulation target in the right hemisphere of the brain based on functional neuroimaging data; and
applying electromagnetic stimulation to the neurostimulation target.

2. The method of claim 1, further comprising measuring one or more symptoms experienced in addition to anxiety from the subject, and wherein the determination of the neurostimulation target is based on the one or more symptoms in addition to functional neuroimaging data.

3. The method of claim 1, wherein the anxiety disorder is generalized anxiety disorder.

4. The method of claim 1, wherein the region of interest comprises a deep brain structure and the neurostimulation target comprises a cortical region of the brain.

5. The method of claim 4, wherein the deep brain structure is the amygdala.

6. The method of claim 4, wherein the cortical region is the dorsolateral prefrontal cortex.

7. The method of claim 1, wherein the region of interest comprises a brain network associated with the anxiety disorder.

8. The method of claim 7, wherein the brain network comprises the dorsal attention network.

9. The method of claim 7, wherein the brain network comprises the anxiosomatic circuit.

10. The method of claim 2, wherein the one or more symptoms experienced in addition to anxiety is selected from the group consisting of anhedonia, depression, dysphoria, panic, fear, fatigue, insomnia, obsessive-compulsiveness, or combinations thereof

11. The method of claim 1, further comprising obtaining one or more clinical factors about the subject, and identifying the neurostimulation target based on the one or more symptoms, functional neuroimaging data, and one or more clinical factors.

12. The method of claim 11, wherein the one or more clinical factors is selected from the group consisting of duration of anxiety, level of anxiety, treatment history for an anxiety disorder, family history of an anxiety disorder, family history of a depressive disorder, post-traumatic stress disorder, an eating disorder, fibromyalgia, demographic information, or combinations thereof

13. The method of claim 1, wherein the functional neuroimaging data describes functional connectivity within the brain.

14. The method of claim 13, wherein determining the neurostimulation target comprises dividing the region of interest into a plurality of sub-parcels, and determining a peak connectivity site for each of the plurality of parcels based on the one or more symptoms and functional neuroimaging data from the subject.

15. The method of claim 14, wherein the region of interest is divided into between 2 and 100 sub-parcels.

16. The method of claim 14, wherein the peak connectivity site is characterized by a pattern of neuronal activity having a high degree of correlation to a pattern of neuronal activity in the corresponding sub-parcel.

17. The method of claim 14, wherein the peak connectivity site is characterized by a pattern of neuronal activity having a high degree of anti-correlation to a pattern of neuronal activity in the corresponding sub-parcel.

18. The method of claim 14, wherein determining the neurostimulation target comprises selecting a subset of the plurality of peak connectivity sites that cluster within a common neuroanatomical region, and designating at least a portion of the common neuroanatomical region as the neurostimulation target.

19. The method of claim 14, wherein determining the neurostimulation target comprises:
assigning, for each of a plurality of potential target locations, a degree of connectivity to each of the plurality of sub-parcels;
calculating, for each of the plurality of potential target locations, an overall degree of connectivity based on a numerical average of the degree of connectivity to each of the plurality of sub-parcels; and
selecting one or more potential target locations having an overall degree of connectivity above a predetermined threshold as the neurostimulation target.

20. The method of claim 1, wherein applying electromagnetic stimulation comprises applying transcranial magnetic stimulation.

21. The method of claim 1, wherein the functional neuroimaging data comprises functional magnetic resonance imaging (fMRI) data.

22. A system for treating an anxiety disorder comprising:
a transcranial magnetic stimulation coil;
a data module configured to receive data comprising functional neuroimaging data of a brain of a subject, wherein the functional neuroimaging data describes neuronal activation within the brain; and
a targeting module comprising a memory storing a set of instructions, and one or more processors responsive to the set of instructions that are configured to determine a neurostimulation target in the right hemisphere of the brain based on functional neuroimaging data.

23-42. (canceled)
